(19) United States
(12) Patent Application Publication
(10) Pub. No.: US 2025/0177317 A1
(43) Pub. Date: Jun. 5, 2025

(54) SELECTIVE AGONISM OF SPECIFIC PATTERN RECOGNITION RECEPTORS IN mRNA CONSTRUCTS FOR SAFE, EFFECTIVE AND DURABLE VACCINES

(71) Applicant: Celestial Therapeutics, Inc., Irvine, CA (US)

(72) Inventor: Ajay GUPTA, Irvine, CA (US)

(21) Appl. No.: 18/703,925

(22) PCT Filed: Oct. 31, 2022
(86) PCT No.: PCT/US2022/048492

§ 371 Date: Apr. 23, 2024

Related U.S. Application Data
(60) Provisional application No. 63/273,988, filed on Oct. 31, 2021.

(51) Int. Cl. A61K 9/51; A61K 9/127

(52) U.S. Cl. CPC A61K 9/5123; A61K 9/127

(57) ABSTRACT

A lipid nanoparticle based composition and method for making the same is provided. The lipid nanoparticle based composition includes an in-vitro transcribed (IVT) mRNA molecule containing (a) a 5′ cap structure, (b) a coding region encoding an antigen polypeptide, (c) an immunostimulatory RNA sequence that activates RIG-I, (d) a poly (A) tail, and (e) a TLR antagonist based on a phospholipid.

### CROSS-REFERENCE TO RELATED APPLICATION

This application claims the benefit of priority from U.S. Provisional Application No. 63/273,988, filed Oct. 31, 2021, having the same inventor and the same title, and which is incorporated herein by reference in its entirety.

### FIELD OF THE DISCLOSURE

The present disclosure relates generally to mRNA vaccines, and more specifically to methods for generating safe, effective and durable vaccines and therapeutics through selective agonism of specific pattern recognition receptors in self-adjuvanting mRNA constructs, and to compositions made by such methods.

### BACKGROUND OF THE DISCLOSURE

Effective in vitro-transcribed (IVT) mRNA vaccines must express an antigen and elicit a robust immune response to the expressed antigen in a safe and a durable fashion. While IVT mRNA stability and protein expression have been greatly improved in recent years, eliciting a balanced, safe and effective immune response to IVT mRNA remains a challenge. Kariko et al. [Kariko, K.; Buckstein, M.; Ni, H. and Weissman, D.; Suppression of RNA recognition by Toll-like Receptors: The impact of nucleoside modification and the evolutionary origin of RNA; Immunity, 2005, 23, 166-175] have previously demonstrated that both natural RNA (isolated from mammalian and bacterial cells) and IVT RNA activate dendritic cells (DCs) mediated via multiple Toll Like Receptors (TLRs), especially TLR3, TLR7 or TLR8. This activation of DCs was shown to be reduced or eliminated when natural nucleosides were substituted with modified nucleosides in the RNA construct such as m5C, m6A, m5U, pseudouridine or 2′-O-methyl-U (1).

Pfizer/BionTech and Moderna have utilized modified nucleosides in their mRNA vaccines against SARS-CoV-2 and have demonstrated that the use of modified nucleosides not only suppresses the immunogenicity of the mRNA constructs but also leads to a robust immunogenic response against the coronavirus. This robust response is due to robust translation of mRNA into the spike protein antigen, thus producing a favorable neutralizing antibody and T-cell response. The Pfizer/BionTech vaccine was administered at 30 micrograms per dose, while the Moderna vaccine was administered at 100 micrograms per dose, both of which are considered relatively high doses.

Two issues have emerged with these vaccines. First of all, some vaccine recipients have suffered side effects from the vaccines. In particular, some vaccine recipients (especially young males) have exhibited myocarditis and pericarditis, while others have suffered neurological functional disorders. A few female recipients of the vaccines have also suffered disruption of menstrual cycles.

Secondly, even though the Pfizer/BionTech and Moderna vaccines are administered at relatively high dosages, the adaptive immune response of these vaccines is not durable, thus requiring frequent boosters. In particular, the efficacy of these vaccines wanes rather quickly, thus requiring a 3rd dose as a booster 6-8 months after administration of the 2nd dose. The Curevac mRNA vaccine for SARS-CoV-2, which uses natural nucleosides, can be used at much lower doses, but performed poorly in phase-3 clinical trials.

### SUMMARY OF THE DISCLOSURE

In one aspect, a lipid nanoparticle based composition is provided which comprises an in-vitro transcribed (IVT) mRNA molecule containing (a) a 5′ cap structure, (b) a coding region encoding an antigen polypeptide, (c) an immunostimulatory RNA sequence that activates RIG-I, (d) a poly (A) tail, and (e) a TLR antagonist based on a phospholipid.

In another aspect, a method for treating or preventing an infection or cancers is provided. The method comprises (a) determining that a subject is suffering from cancer or has contracted, or is in danger of contracting, an infection; and (b) administering to the subject a lipid nanoparticle based composition, comprising an in-vitro transcribed (IVT) mRNA molecule containing (i) a 5′ cap structure, (ii) a coding region encoding an antigen polypeptide, (iii) an immunostimulatory RNA sequence that activates RIG-I, (iv) a poly (A) tail, and a TLR antagonist based on a phospholipid.

### BRIEF DESCRIPTION OF THE DRAWINGS

FIG. 1 is a drawing illustrating differences between safety and efficacy of various approaches to generate vaccines (source: http://www.javireport.org).

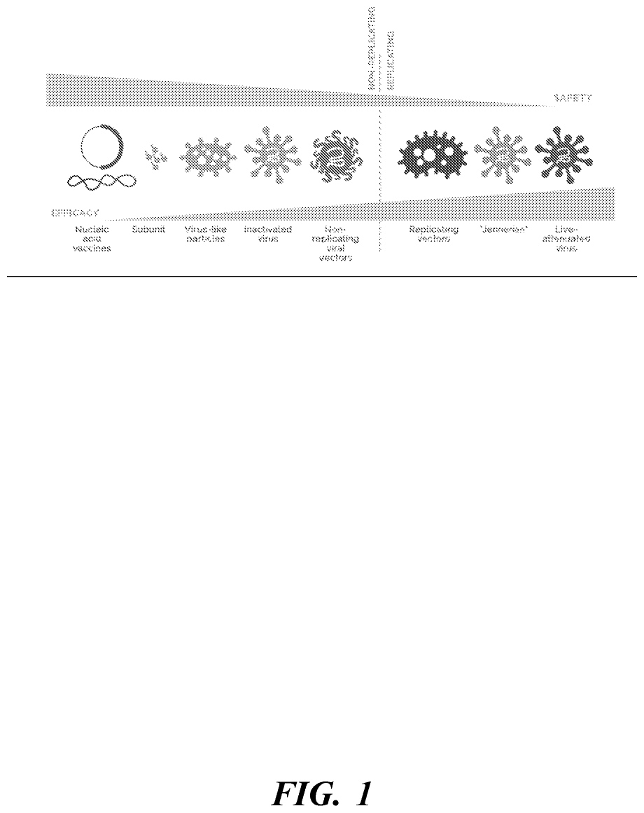

### DETAILED DESCRIPTION

U.S. Pat. No. 10,736,957 (Chan et al.) discloses that encoding short noncoding immunostimulatory RNA (isRNA) sequences within a longer mRNA molecule can enhance innate immune responses to IVT mRNA in vitro, leading to increased interferon and cytokine expression and upregulation of antigen presentation. IVT mRNA containing an isRNA sequence that specifically activates the pattern-recognition receptor RIG-I was found to induce higher levels of type-I interferon (IFN) expression and to increase apoptosis when transfected into cancer cells.

It has now been found that it may be possible to generate safer, more efficacious and more durable mRNA vaccines and therapeutics by using natural nucleosides in a self-adjuvanting mRNA construct, but selectively agonizing a specific pattern recognition receptor (PRR) such as RIG-I while selectively antagonizing multiple TLRs using a broad spectrum TLR antagonist. Suitable TLR agonists (TLRAs) for this purpose may include, for example, 1-palmitoyl-2-oleoyl-sn-glycero-3-phospho-(l′-rac-glycerol) (sodium salt) (POPG) or analogs thereof. These vaccines and therapeutics may be applied against a variety of diseases including, but not limited to, infectious diseases, cancers, and the like. The broad spectrum TLR antagonist may be simply added to the vaccine composition, which is preferably a lipid nanoparticle (LNP) based formulation. Liposomal formulations of TLRAs can be prepared separately and added in various ratios to the LNP-based mRNA formulations. Preferably, the ratio of liposome to the LNP is within the range of 1000:1 to 1:1000 Liposome:LNP on a v/v basis, and the concentration of the TLR antagonist in the liposomal formulation is preferably within the range of 1 ng/ml to 100 mg/ml. Besides mixing the liposomes into the mRNA formulated into the LNPs, the TLR antagonist may also be encapsulated into the LNPs. Preferably, the ratio of mRNA:TLRA is within the range of 1000:1 to 1:1000 mRNA:TLRA on a wt/wt or mole/mole basis.

In a preferred embodiment, an mRNA vaccine is provided in the form of an LNP-based composition. The composition preferably includes an in-vitro transcribed (IVT) mRNA molecule containing a 5′ cap structure, a coding region encoding an antigen polypeptide, an immunostimulatory RNA sequence that activates RIG-I, a poly (A) tail, and a TLR antagonist based on a phospholipid. In some embodiments, the composition further comprises at least one anionic lipid or related compound, wherein the amount of the anionic lipid or related compound is effective to antagonize at least one TLR, and wherein the anionic lipid or related compound has a hydrophobic portion; a negatively charged portion; and an uncharged, polar portion.

Various anionic lipids or related compounds may be utilized in the compositions disclosed herein. Preferably, the anionic lipid or related compound is selected from the group consisting of unsaturated phosphatidylglycerols, unsaturated phosphatidylinositols, saturated short chain phosphatidylglycerols, saturated short chain phosphatidylinositols, anionic sphingolipids, anionic glycerolipids, unsaturated lyso-phosphatidylglycerols, saturated lyso-phosphatidylglycerols, unsaturated lyso-phosphatidylinositols, saturated lyso-phosphatidylinositols, and derivatives of the foregoing. More preferably, the anionic lipid is selected from the group consisting of unsaturated phosphatidylglycerols, unsaturated phosphatidylinositols, saturated short chain phosphatidylglycerols, saturated short chain phosphatidylinositols, and derivatives of the foregoing. Most preferably, the anionic lipid is an unsaturated phosphatidylglycerol or a derivative thereof, palmitoyl-oleoyl-phosphatidylglycerol (POPG) or a derivative thereof, or an unsaturated phosphatidylinositol or a derivative thereof.

The compositions disclosed herein may be effective against viruses associated with various toll-like receptors (TLRs). Preferably, the composition is effective against a coronavirus infection associated with a toll-like receptor (TLR) selected from the group consisting of TLR1, TLR2, TLR3, TLR4, TLR6, TLR7, TLR8, TLR9 and TLR10.

The IVT RNA sequence of the compositions disclosed herein may have various cap structures. Preferably, the IVT RNA sequence has a 5′ cap structure comprising a synthetic cap structure selected from the group consisting of 3′-O-Me-m7G(5′)ppp(5′)G, m7G(5′)ppp(5′)G and G(5′)ppp(5′)G. The IVT RNA molecule also preferably comprises of a linker sequence between the coding region encoding the antigen polypeptide and the immunostimulatory RNA sequence that activates RIG-I. The linker sequence preferably contains a poly(A) binding protein. Details of such a binding protein may be found, for example, in [Kahvejian, A., Svitkin, Y. V., Sukarieh, R., M'Boutchou, M.-N., Sonenberg, N., 2005. Mammalian poly(A)-binding protein is a eukaryotic translation initiation factor, which acts via multiple mechanisms. Genes & Development 19, 104-113. doi:10.1101/gad.1262905], which is incorporated herein by reference in its entirety.

The above description of the present invention is illustrative, and is not intended to be limiting. It will thus be appreciated that various additions, substitutions and modifications may be made to the above described embodiments without departing from the scope of the present invention. Accordingly, the scope of the present invention should be construed in reference to the appended claims. For convenience, some features of the claimed invention may be set forth separately in specific dependent or independent claims. However, it is to be understood that these features may be combined in various combinations and subcombinations without departing from the scope of the present disclosure. By way of example and not of limitation, the limitations of two or more dependent claims may be combined with each other without departing from the scope of the present disclosure.

1. A lipid nanoparticle based composition, comprising:
an in-vitro transcribed (IVT) mRNA molecule containing
(a) a 5′ cap structure,
(b) a coding region encoding an antigen polypeptide,
(c) an immunostimulatory RNA sequence that activates RIG-I,
(d) a poly (A) tail, and
(e) a TLR antagonist based on a phospholipid.

2. The composition of claim 1, further comprising at least one anionic lipid, wherein the amount of the anionic lipid is effective to antagonize at least one TLR, and wherein the anionic lipid has:
a. a hydrophobic portion;
b. a negatively charged portion; and
c. an uncharged, polar portion.

3. The composition of claim 1, wherein the anionic lipid is selected from the group consisting of unsaturated phosphatidylglycerols, unsaturated phosphatidylinositols, saturated short chain phosphatidylglycerols, saturated short chain phosphatidylinositols, anionic sphingolipids, anionic glycerolipids, unsaturated lyso-phosphatidylglycerols, saturated lyso-phosphatidylglycerols, unsaturated lyso-phosphatidylinositols, saturated lyso-phosphatidylinositols, and derivatives of the foregoing.

4. The composition of claim 1, where the TLR antagonist is a liposomal formulation.

5. The composition of claim 4, wherein the ratio of the liposomal formulation to the lipid nanoparticle based composition is within the range of 1000:1 to 1:1000 on a v/v basis.

6. The composition of claim 5, wherein the concentration of the TLR antagonist (TLRA) in the liposomal formulation is within the range of 1 ng/ml to 100 mg/ml.

7. The composition of claim 6, wherein the TLR antagonist is encapsulated into the LNPs, and where the ratio of mRNA:TLRA is within the range of 1000:1 to 1:1000 on a wt/wt or mole/mole basis.

8. The composition of claim 7, where the ratio of liposome to LNP is within the range of 1000:1 to 1:1000 on a v/v basis.

9. The composition of claim 1, wherein the anionic lipid is selected from the group consisting of unsaturated phosphatidylglycerols, unsaturated phosphatidylinositols, saturated short chain phosphatidylglycerols, saturated short chain phosphatidylinositols, and derivatives of the foregoing.

10. The composition of claim 1, wherein the anionic lipid is an unsaturated phosphatidylglycerol or a derivative thereof.

11. The composition of claim 1, wherein the anionic lipid is palmitoyl-oleoyl-phosphatidylglycerol (POPG) or a derivative thereof.

12. The composition of claim 1, wherein the anionic lipid is an unsaturated phosphatidylinositol or a derivative thereof.

13. The composition of claim 1, wherein the composition is effective against a coronavirus infection associated with a toll-like receptor (TLR) selected from the group consisting of TLR1, TLR2, TLR3, TLR4, TLR6, TLR7, TLR8, TLR9 and TLR10.

14. The composition of claim 1, wherein the IVT RNA sequence has a 5′ cap structure comprising a synthetic cap structure selected from the group consisting of 3′-O-Me-m7G(5′)ppp(5′)G, m7G(5′)ppp(5′)G and G(5′)ppp(5′)G.

15. The composition of claim 1, wherein the IVT RNA molecule comprises of a linker sequence between the coding region encoding the antigen polypeptide and the immunostimulatory RNA sequence that activates RIG-I.

16. The composition of claim 1, wherein the IVT RNA molecule has the linker sequence containing poly(A).

17. A method for treating or preventing an infection or treating a cancer, comprising:
determining that a subject is suffering from cancer or has contracted, or is in danger of contracting, an infection; and
administering to the subject a lipid nanoparticle based composition, comprising an in-vitro transcribed (IVT) mRNA molecule containing
(a) a 5′ cap structure,
(b) a coding region encoding an antigen polypeptide,
(c) an immunostimulatory RNA sequence that activates RIG-I,
(d) a poly (A) tail, and
(e) a TLR antagonist based on a phospholipid to prevent or to treat diseases.

18. The method of claim 17, further comprising at least one anionic lipid, wherein the amount of the anionic lipid is effective to antagonize at least one TLR, and wherein the anionic lipid has:
a. a hydrophobic portion;
b. a negatively charged portion; and
c. an uncharged, polar portion.

19. The method of claim 17, wherein the anionic lipid is selected from the group consisting of unsaturated phosphatidylglycerols, unsaturated phosphatidylinositols, saturated short chain phosphatidylglycerols, saturated short chain phosphatidylinositols, anionic sphingolipids, anionic glycerolipids, unsaturated lyso-phosphatidylglycerols, saturated lyso-phosphatidylglycerols, unsaturated lyso-phosphatidylinositols, saturated lyso-phosphatidylinositols, and derivatives of the foregoing.

20. The method of claim 17, where the TLR antagonist is a liposomal formulation.

21.-32. (canceled)
